(19) United States
(12) Patent Application Publication
(10) Pub. No.: US 2011/0071399 A1
(43) Pub. Date: Mar. 24, 2011

(54) ULTRASONIC PROBE

(76) Inventors: Shengli Tang, Shenzhen (CN); Xunhua Xiao, Shenzhen (CN)

(21) Appl. No.: 12/886,373

(22) Filed: Sep. 20, 2010

(30) Foreign Application Priority Data
Sep. 21, 2009 (CN) 200910190464.2

(51) Int. Cl. A61B 8/14

(52) U.S. Cl. 600/459

(57) ABSTRACT

An ultrasonic probe includes a base; a motor fixedly connected to the base; a driving wheel connected to the output of the motor and driven by the motor; a driven wheel rotationally connected to the base with a shaft; a transducer fixedly connected to the driven wheel for emitting and receiving ultrasonic echoes; two ropes, both ends of each rope having a connection end respectively; one connection end on one end of each rope respectively connected to the driving wheel and another connection end on another end of each rope respectively connected to the driven wheel; and at least one elastic part through which the connection end of at least one end of at least one rope connected to the driving wheel or the driven wheel in a buffering manner, wherein, one end of the elastic part connected to at least one connection end of the rope, and another end of the elastic part connected to the driving wheel or the driven wheel.

### TECHNICAL FIELD

The present disclosure relates generally to ultrasonic imaging and more particularly to a 3D mechanical scanning probe for medical ultrasonic imaging.

### SUMMARY OF THE INVENTION

Various embodiments of a probe for use in medical ultrasonic imaging are disclosed.

### BRIEF DESCRIPTION OF THE DRAWINGS

FIG. 1 is an axonometric view of an embodiment of the present disclosure;

FIG. 2 is an exploded perspective view of a transducer and a driven wheel;

FIG. 3 is an exploded perspective view of a motor and a driving wheel;

FIG. 4 is a perspective view of an embodiment of the present disclosure schematically illustrating hiding the base after the probe has been mounted;

FIG. 5 is a perspective view of an embodiment of the present disclosure schematically illustrating the connection of the ropes;

FIG. 6 is a sectional view of an embodiment of the present disclosure illustrating the probe as it passes through the centre line of the shaft;

FIG. 7 is an axonometric drawing of one side of an embodiment of the present disclosure;

FIG. 8 is an axonometric drawing of another side relative to FIG. 7;

FIG. 9 is an axonometric drawing of an elastic component;

FIG. 10 is a perspective view schematically illustrating an embodiment of the present disclosure;

FIG. 11 is a perspective view of an embodiment of the present disclosure schematically illustrating the winding of the ropes;

FIG. 12 is a perspective view schematically illustrating an embodiment of the present disclosure;

FIG. 13 is an axonometric view of an embodiment of the present disclosure;

FIG. 14 is a perspective view of an embodiment of the present disclosure schematically illustrating hiding the base;

FIG. 15 is a perspective view of an embodiment of the present disclosure schematically illustrating the connection of the ropes;

FIG. 16 is a perspective view schematically illustrating an embodiment of the present disclosure;

FIG. 17 is a perspective view of an embodiment of the present disclosure schematically illustrating the connection of the ropes;

FIG. 18 is an exploded perspective view of an embodiment of the present disclosure illustrating the connection of the ropes;

FIG. 19 is a perspective view schematically of an embodiment of the present disclosure illustrating the connection of the ropes;

FIG. 20 is an exploded perspective view of an embodiment of the present disclosure illustrating the connection of the ropes;

FIG. 21 is a part sectioned view of an embodiment of the present disclosure illustrating the connection between the rope and the driven wheel;

FIG. 22 is a perspective view of an embodiment of the present disclosure schematically illustrating the connection of the ropes;

FIG. 23 is a perspective view of an embodiment of the present disclosure schematically illustrating the connection of the ropes;

FIG. 24 is an exploded perspective view of an embodiment of the present disclosure illustrating the connection of the ropes; and

FIG. 25 is a perspective view of an embodiment of the present disclosure schematically illustrating the connection of the ropes.

### DETAILED DESCRIPTION

An ultrasonic probe for a 3D ultrasonic imaging system is generally referred to as a 3D mechanical probe, which internally has a step motor as a driving power source to drive a transducer to swing at a certain angle. At every swing angle, the 3D mechanical probe works like the conventional probe by emitting ultrasound waves and receiving echo information about human tissue. As a result, ultrasonic imaging of human tissues may be obtained at every swing angle without sliding or swinging a probe on a human body by a doctor.

The 3D mechanical probe needs to transmit the motion of the step motor to the transducer. The normal method to transmit the motion to the transducer is using ropes. In such a case, the ropes should be tightened. However, the motion of the step motor is not smooth as the speed or direction can have sudden changes when in use, which will create an impact on the rope or on the transducer. Thus it is necessary to cushion the impact.

The structure of the rope connecting device in the existing ultrasonic probe is complicated, expensive, and difficult to assemble. Therefore, an easily mounted ultrasonic probe with a simple structure is needed to provide continuous power to keep the rope tight and cushion the impact when in use.

According to one aspect of the present disclosure, an ultrasonic probe includes a base; a motor fixedly connected to the base; a driving wheel connected to the output of the motor and driven by the motor; a driven wheel rotationally connected to the base with a shaft; a transducer fixedly connected to the driven wheel for emitting and receiving ultrasonic echoes; two ropes, both ends of each rope having a connection end respectively, one connection end on one end of each rope respectively connected to the driving wheel and another connection end on another end of each rope respectively connected to the driven wheel; and at least one elastic part through which the connection end of at least one end of at least one rope is connected to the driving wheel or the driven wheel in a buffering manner, wherein one end of the elastic part is connected to at least one connector on one end of the ropes, and another end of the elastic part is connected to the driving wheel or the driven wheel.

Referring generally to FIGS. 1-6, an ultrasonic probe may include a base 1, a transducer 2, a driven wheel 3, a support wheel 4, a transducer base 5, a shaft 6, a motor 11, a motor support 12, a driving wheel 14, two ropes 17, 18, and two springs 16a-b.

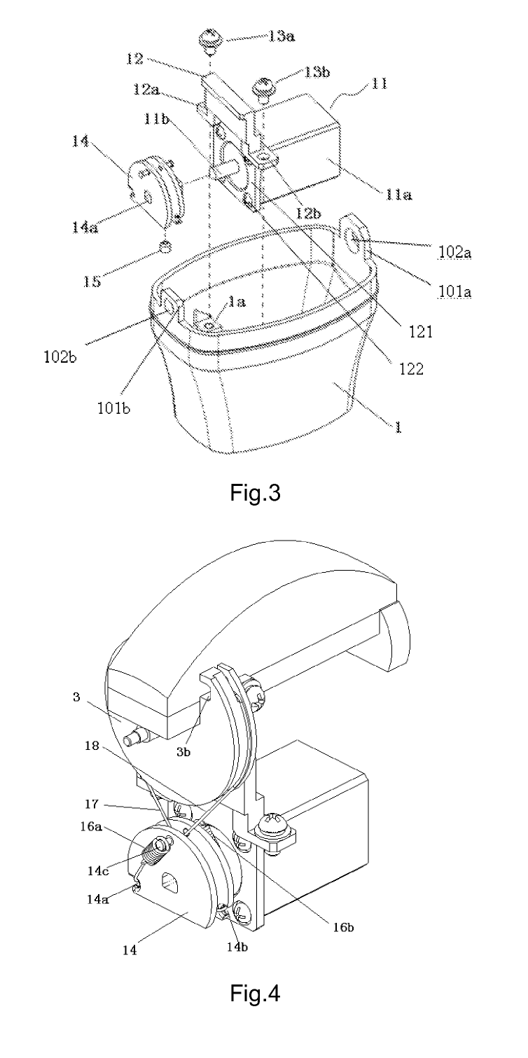

As shown in FIG. 3, the base 1 may be embodied as a hollow receptacle, within which there is a screw hole 1a and another screw hole on the symmetrical side of the screw hole 1a (the screw hole is not shown at the position corresponding to la on the dotted line corresponded to screw 13b in FIG. 3, which is numbered as 1b). In one embodiment, two fixing flats respectively stretched out of both sides of the motor support 12, on which mounting holes 12a and 12b are formed. To fix the motor support 12 on the base 1, the screw 13a matches the screw hole 1 a through the mounting hole 12a, and the screw 13b matches the screw hole 1 b through the mounting hole 12b. With four screws 122, the motor 11 is installed and fixed to a mounting plate 121 stretched out of the motor support.

In one embodiment, the location of the motor 11 and the motor support 12 may be adjusted in a certain range in the vertical direction shown in the FIG. 3 by turning the screws 13a and 13b. As the locations of the driven wheel 3, the shaft 6 and the transducer 2 are above those of the driving wheel 14, the motor support 12 and the motor 11 when mounted (the vertical direction shown in FIG. 3), the distance between the driving wheel 14 and the driven wheel 3 in a small range may be adjusted by turning the screws 13a and 13b and the tightness of the rope may be adjusted thereby.

The driving wheel connected to the output of the motor can make the motor turn the driving wheel in one embodiment, wherein the driving wheel connected to the output of the motor is not only the driving wheel connected to the output of the motor directly, but also the driving wheel indirectly connected to the output of the motor via other components, which can make the motor drive the driving wheel. For example, the driving wheel may be directly fixed on the output of the motor, or the driving wheel may be connected to the output of the motor via a driving belt. In the illustrated embodiment, the driving wheel is directly fixed on the output of the motor. As shown in the FIG. 3, the output shaft 11 b of the motor 11 extends through a round hole on the mounting plate 121, penetrates the mounting hole 14a of the driving wheel 14, and fixes the driving wheel 14 with a fastening screw 15. The motor 11 drives the driving wheel 14 by the output shaft 11b thereby.

As shown in FIG. 1 and FIG. 3, two extending panels 101a and 101b are extended upward along both sides of the base 1 respectively. An axle hole 102a is formed on the extending panel 101a and another axle hole 102b on the panel 101b. The inside of each axle hole has a bearing (not shown). A revolute pair is made by one end of the shaft 6 rotationally connected to the extending panel 101a with the bearing of the axle hole 102a, and another revolute pair is made by another end of the extending panel 101 b with the bearing of the hole 102b.

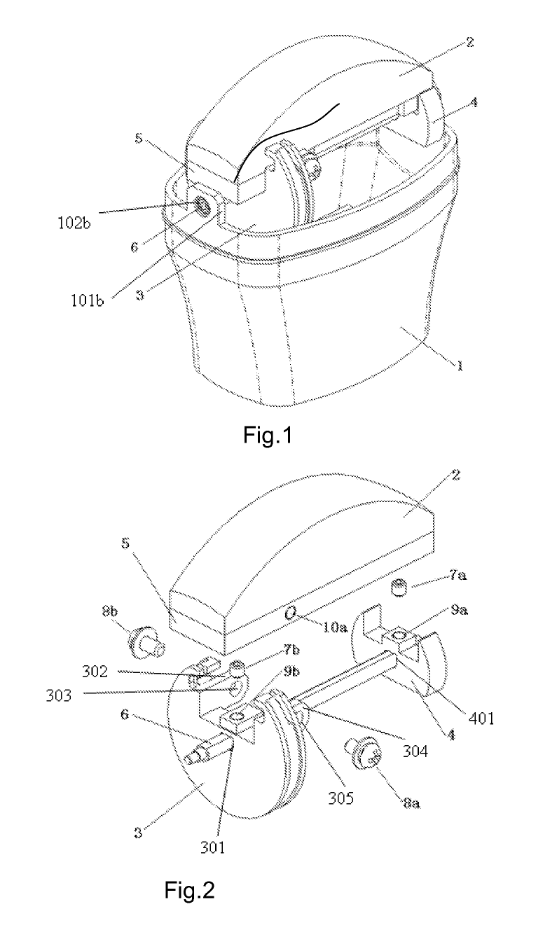

As shown in the FIG. 2, the shaft 6 passes through a mounting hole 301 on the rotating axis of the driven wheel 3 and a mounting hole 401 on the rotating axis of the support wheel 4. The fastening screw 7b screws in the fastening screw hole 9b formed on the driven wheel 3 and its one end against the shaft 6 tightly to make the driven wheel 3 fix on the shaft 6. The fastening screw 7a screws in the fastening screw hole 9a formed on the support wheel 4 and its one end against the shaft 6 tightly to make the support wheel 4 fix on the shaft 6.

The transducer 2 may be fixed on the transducer base 5 using various conventional methods not discussed in detail here. Two grooves are respectively formed on the upper side of the driven wheel 3 and that of the support wheel 4. The transducer 2 and the transducer base 5 may be engaged with the grooves of the driven wheel 3 and the support wheel 4 respectively. Two holding sheets 302 and 304 of the driven wheel 3, which are respectively perpendicular to the surface of the wheel, are extended out of the two opposite sides of the groove. Two mounting holes 303 and 305 are respectively formed on the holding sheet 302 and 304. A screw hole 10a is formed on one side of the transducer base 5 and another screw hole on the symmetrical position of another side (not shown in the figure). In one embodiment, the fastening screw 8a screws in the screw hole 10a of the transducer base 5 through the mounting hole 305 of the holding sheet 304, and the fastening screw 8b screws in the screw hole of the transducer base 5 (the screw hole not shown in the figure) through the mounting hole 303 of the holding sheet 302. In this way, the transducer base 5 and the transducer 2 may be fixedly connected to the driven wheel 3 with the fastening screw 8a and 8b, so that the transducer base 5 and transducer 2 may be swung around the shaft 6 with the driven wheel 3.

The springs or ropes are connected to the driving wheel with a joint structure provided by the driving wheel. The joint structure may be any suitable structure as long as the springs may be connected thereto, such as pin, hook, bolt, groove, slot, etc. The pin is used as the joint structure in the embodiment. As shown from FIG. 4 to FIG. 6, a pin 14c is fixedly attached to the lateral surface 141 of the driving wheel 14 and another pin 14d fixedly attached to another lateral surface 142. A concave groove 144 is formed on the circum surface 143 of the driving wheel 14 along the circumferential direction. A gap 14a formed on one side of the lateral surface 141 connects the lateral surface 141 to the concave groove 144, and another gap 14b formed on another side of the lateral surface 142 corresponded to the gap 14a connects the lateral surface 142 to the concave groove 144.

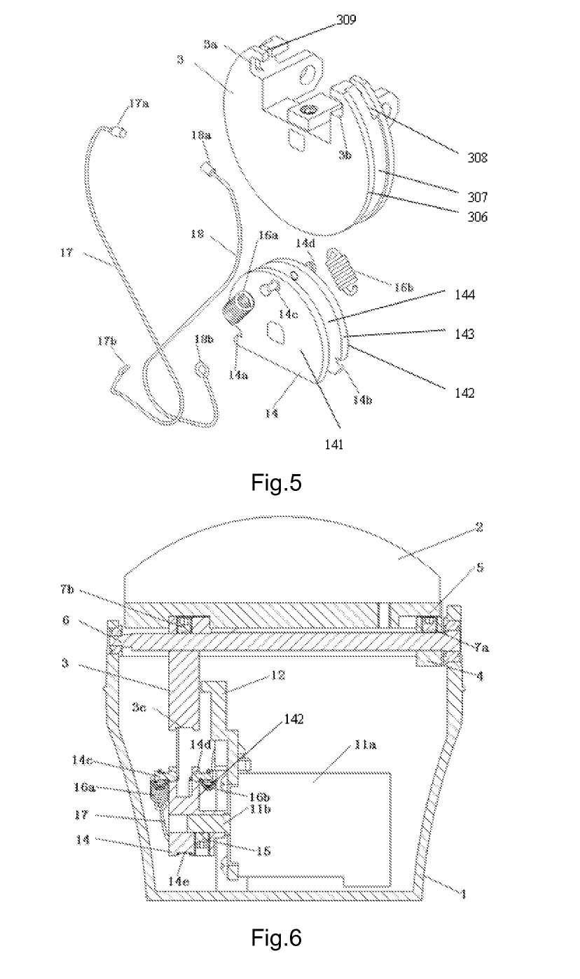

In one embodiment, the springs or ropes are connected to the driven wheel with joint structures provided by the driven wheel. The joint structures may be any suitable structure as long as the springs may be connected thereto, such as pin, hook, bolt, groove, slot, etc. The groove is used as the joint structures in the illustrated embodiment. As shown in FIG. 5, a concave groove 307 is formed on the circum surface 306 of the driven wheel 3 along the circumferential direction. Two joint structures 3a and 3b are arranged on the upper of the two sides of the concave groove 307. A slot 309 which is formed on the joint structure 3a connects the concave groove 307 to the joint structure 3a, and another slot 308 which is formed on the joint structure 3b connects the concave groove 307 to the joint structure 3b.

Two hooks are respectively formed at the two ends of the spring 16a, wherein one hook at one end hooks the pin 14c, and another hook at another end hooks the connection end 17b of one end of the rope 17. (In the illustrated embodiment, the connection end 17b of the rope 17 is a ring.) The rope 17 passes the gap 14a into the concave groove 144, winds the driving wheel 14 along the concave groove for a certain distance, enters the concave groove 307 of the driven wheel 3, winds along the concave groove 307 for a certain distance, and enters the joint structure 3a through the gap 309. Another connection end 17a on another end of the rope 17 is clamped to the joint structure 3a (the connection end 17a is a knot in the embodiment).

Similarly, two hooks are respectively formed at the two ends of the spring 16b, wherein one hook at one end hooks the pin 14d, and another hook hooks the connection end 18b of one end of the rope 18 (the connection end 18b of the rope 18 is a ring in the embodiment). The rope 18 passes the gap 14b into the concave groove 144, winds the driving wheel 14 along the concave groove for a certain distance, enters the concave groove 307 of the driven wheel 3, winds along the concave groove 307 for a certain distance, and enters the joint structure 3b through the gap 308. Another connection end 18a on another end of the rope 18 is clamped to the joint structure 3b (the connector 18b is a knot in this embodiment). The winding direction of the rope 18 is opposite to that of the rope 17 in the concave groove 144 of the driving wheel 14 and the groove 307 of the driven wheel 3.

When the probe works, the motor 11 turns, thus driving the driving wheel 14. The driving wheel 14 transmits the motion to the driven wheel 3 with the rope 17 and 18, thus driving the driven wheel 3 and the shaft 6. The transducer 2 is fixedly connected to the shaft 6, thus driving the transducer 2 thereby. The rotation direction of the motor 11 changed repeatedly can realize the transducer 2 swing within a certain range.

The springs 16a and 16b may keep the ropes tight continuously and perform a shock absorption function in case the impact to the ropes and the driven wheel caused by the motor 11 changes the rotation direction. In addition, with the spring, the demand for the accuracy of rope length may be reduced. The ropes may be wire rope, or may be made of other materials suitable materials. The connection ends of the ropes may be processed with conventional methods, such as die casting, or directly twisted by wire rope manufacturers. The connection ends may be knots, rings or other similar joint parts according to requirements. To reduce the damage to the ropes, the gaps 14a and 14b may be rounded off.

FIG. 6 is a sectional view of one embodiment. The position of the ropes 17 and 18 winding on the driving wheel 14 is 14e, which is a cylindrical surface herein. The position of the ropes 17 and 18 winding on the driven wheel 3 is 3c, which is a part of the cylindrical surface. Tangency point of the ropes and 3c on the cylindrical surface can ensure the ratio of the angle the driving wheel 14 rotated to the angle the driven wheel 3 rotated equal to the diameter ratio of the two cylindrical surfaces.

The ropes on the driving wheel and driven wheel which are kept away from sliding to avoid the rotation angle error caused by the ropes sliding. After the ropes 17 and 18 are connected to and wound around the driving wheel 14 and the driven wheel 3, turning the screws 13a and 13b can enlarge the distance between the driving wheel 14 and the driven wheel 3 to make the ropes tight. Thus no other forms of strength needed to make the springs out of shape.

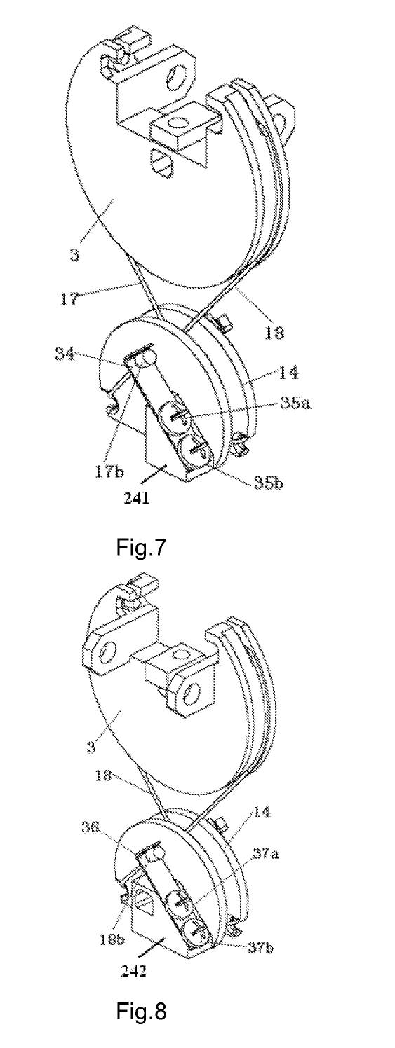

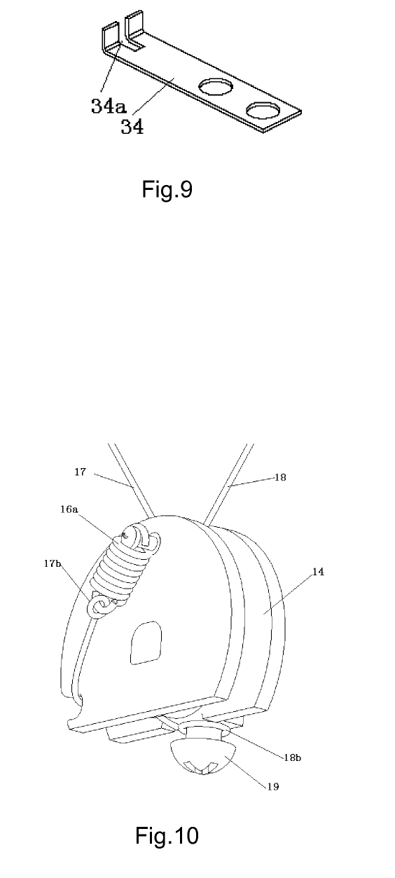

The springs 16a and 16b may be replaced with other elastic parts in the illustrated embodiment. As shown in FIG. 7 to FIG. 9, the springs may be replaced with elastic slices. As shown in FIG. 7 and FIG. 8, two bosses 241 and 242 are formed on the two lateral surfaces of the driving wheel 14 respectively. An elastic slice 34 is fastened to the boss 241 of the driving wheel 14 with the screws 35a and 35b. As shown in FIG. 9, a joint structure 34a is formed on the elastic slice 34 forms. The rope 17, with the connection end 17b (which is a knot in the depicted embodiment) at its one end clamped at the joint structure 34a of the elastic slice 34, passes the gap 14a into the concave groove 144, winds the driving wheel 14 along the concave groove for a certain distance, enters the concave groove 307 of the driven wheel 3, winds along the groove 307 for a certain distance, and enters the joint structure 3a through the gap 309. Another connection end 17a (which is a knot in the depicted embodiment) on another end of the rope 17 is clamped to the joint structure 3a. That is, the winding of the rope 17 and the connection end 17a connected to the driven wheel 3 as described above.

The connection of the rope 18 may be identical to that of the rope 17. As shown in FIG. 8, an elastic slice 36, whose shape is same to the elastic slice 34, is fastened to the boss 242 of the driving wheel 14 with the screw 37a and 37b. The rope 18, with the connection end 18b (which may be a knot) at its one end clamped at the joint structure of the elastic slice 36, passes the gap 14b into the concave groove 144, winds the driving wheel 14 along the groove for a certain distance, enters the concave groove 307 of the driven wheel 3, winds along the groove 307 for a certain distance, and enters the joint structure 3b through the gap 308. Another connection end 18a (which may be a knot) on another end of the rope 18 is clamped to the joint structure 3b. That is, the winding of the rope 18 and the connection end 18a connected to the driven wheel 3 is identical to those described above.

In the illustrated embodiment, the elastic slice 34 may keep the ropes tight continuously and perform a shock absorption function in case the impact on the ropes and the driven wheel caused by the motor 11 changes the rotation direction. In addition, with the elastic slice, the demand for the accuracy of rope length may be reduced. Furthermore, the elastic slice may be easily manufactured.

In various embodiments, two elastic parts (springs or elastic slices), each of which is respectively connected to a rope, may be used. An elastic part connected to a rope and another rope directly connected to driving wheel without any elastic parts is allowed.

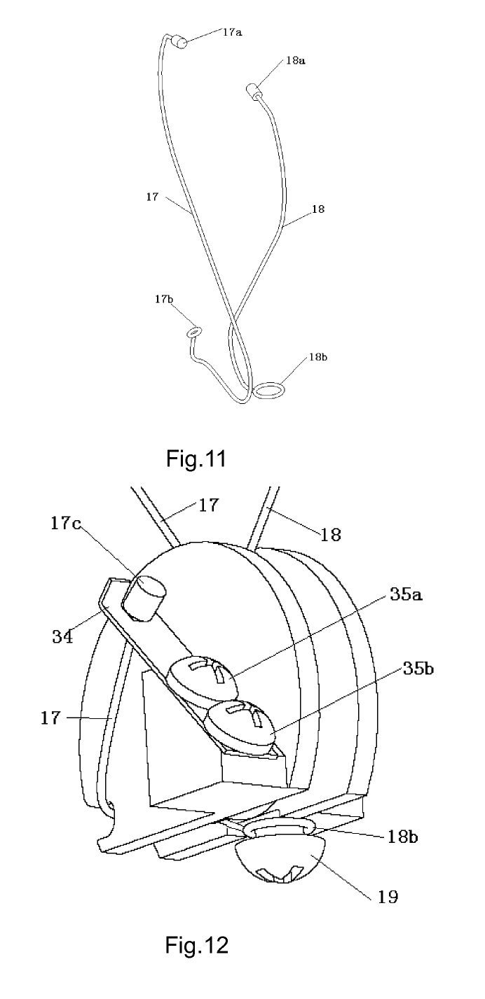

Various embodiments of the present disclosure are shown in FIG. 10 to FIG. 12. In one embodiment, as shown in FIG. 10 and FIG. 11, the driving wheel 14 is provided with a screw 19. The rope 18, with its connection end 18b attached to the screw 19, winds at the position 14e on the driving wheel 14 and the position 3c on the driven wheel 3. The winding in this embodiment is similar to that in the aforesaid embodiments. The rest of the parts in this embodiment are identical to the aforesaid embodiments. In the illustrated embodiment, the two ropes may be tightened by using one spring.

Similarly, FIG. 12 shows an embodiment of the present disclosure, whose structure is mostly as same as the last embodiment. The difference is that, instead of spring being used, an elastic slice, whose construction is identical to the elastic slice described above, is used.

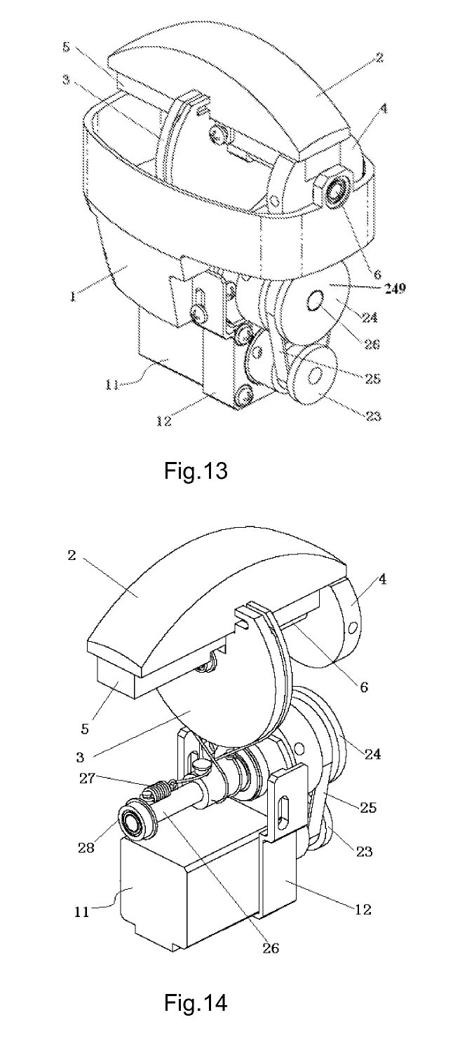

Another embodiment of the present disclosure is shown in FIG. 13 to FIG. 15. As illustrated, the motor 11 is fastened to the base 1 by the motor support 12. A driving timing pulley 23 and a driving wheel 24 may be used (in the present disclosure, the wheel, which directly drives the driven wheel connected to the transducer, is regarded as a driving wheel; in the illustrated embodiment, though the wheel 24 is driven by the driving timing pulley 23, it is still referred to as “driving wheel” for it directly drives the driven wheel 3). The driving timing pulley 23, which is fixedly connected with the output shaft of the motor, drives the driving wheel 24 through the timing belt 25; that is, the driving wheel 24 is connected to the output of the motor 11 by the timing belt 25.

The driving wheel 24 may include a driven timing pulley 249 and a revolving shaft 26. The driven timing pulley 249 is fixedly connected with one end of the revolving shaft 26. The timing belt 25 wraps around the driven timing pulley. The revolving shaft 26 is rotationally connected with the base 1 through a bearing (such as the bearing 28) and may be rotated freely relative to the base 1.

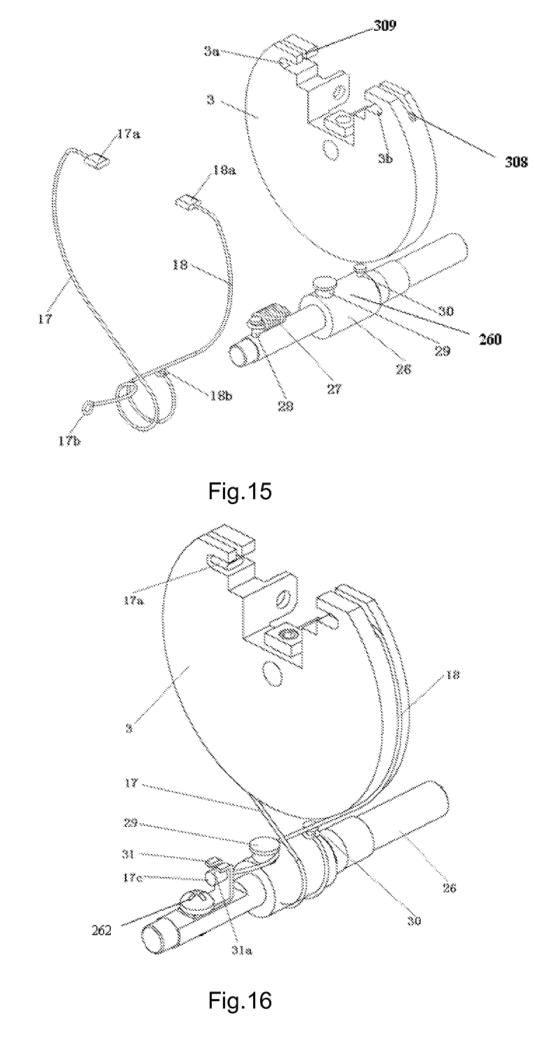

As shown in FIG. 15, a winding area 260 for ropes wound, whose surface shape may be a cylindrical or other curved surface to meet practical requirements, is arranged on the middle part of the revolving shaft 26. A king pin 29 and a link pin 30 are respectively arranged at the two sides of the winding area; a link pin 28 is arranged near the end of the revolving shaft 26. The king pin 29 is located between the link pin 28 and the link pin 30, and further, the distance the king pin 29 to the end of the revolving shaft 26 is larger than that the link pin 28 to the end. The rope 17, whose connection end 17a (which may be a knot) is clamped to the joint structure 3a of the driven wheel 3 through the gap 309, winds the driven wheel 3 for a certain distance, winds the winding area 260 of the revolving shaft 26 for a certain distance, and bypasses the king pin 29 fixedly connected with the revolving shaft 26, and connects to the spring 27 with the connection end 17b (which may be a ring) clamped to the hook ring at one end of the spring 27. Another hook ring at another end of the spring 27 hooks to the link pin 28 fixedly connected with the revolving shaft 26.

The rope 18, whose connection end 18a (which may be a knot) is clamped to the joint structure 3b of the driven wheel 3 through the gap 308 of the driven wheel 3, winds the driven wheel 3 for a certain distance, winds the winding area 260 of the revolving shaft 26 for a certain distance and connects to the revolving shaft 26 by the connection end 18b (which may be a ring) clamped to the link pin 30 which is fixedly connected with the revolving shaft 26. In this way, when the motor 11 turns, it drives the driving timing pulley 23. The revolving shaft 26 of the driving wheel 24, which is turned by the driving timing pulley 23, turns the driven wheel 3 through the ropes 17 and 18.

In the illustrated embodiment, the two ropes are tightened continuously by the tension offered by the spring 27. Furthermore, as one end of the spring 27 is connected with the rope 17 which bypasses the king pin 29, and another end of the spring is connected to the link pin 28 which is located at one side of the king pin 29 along the axis of the revolving shaft 26, the spring 27 is located along the axis of the shaft 26 after installed. Take the axis of the revolving shaft 26 in the probe as the radial direction of the probe and the direction along the transducer 2 to the motor 11 as the axial direction of the probe, the structure in the illustrated embodiment takes up a small space along the axial direction, and meets the requirement of taking up a small space along the axial direction in the probe.

As shown FIG. 16, the spring may be replaced with other elastic parts. For example, an elastic slice 31, which is almost L-shape and provided with a joint structure 31a, may be arranged near one end of the shaft 26. The elastic slice 31 is fixedly connected to the revolving shaft 26 of the driving wheel 24 through a screw 262. The connection end 17a of the rope 17 is clamped in the joint structure 3a of the driven wheel 3, and the connection end 17c is clamped in the joint structure 31a of the elastic slice 31.

Two springs, which are respectively connected to a rope in a buffering manner, may be used in the aforesaid embodiments. One spring is connected to the driving wheel in a buffering manner and another spring to the driven wheel in a buffering manner. In this way, the two ropes may be tighten continuously.

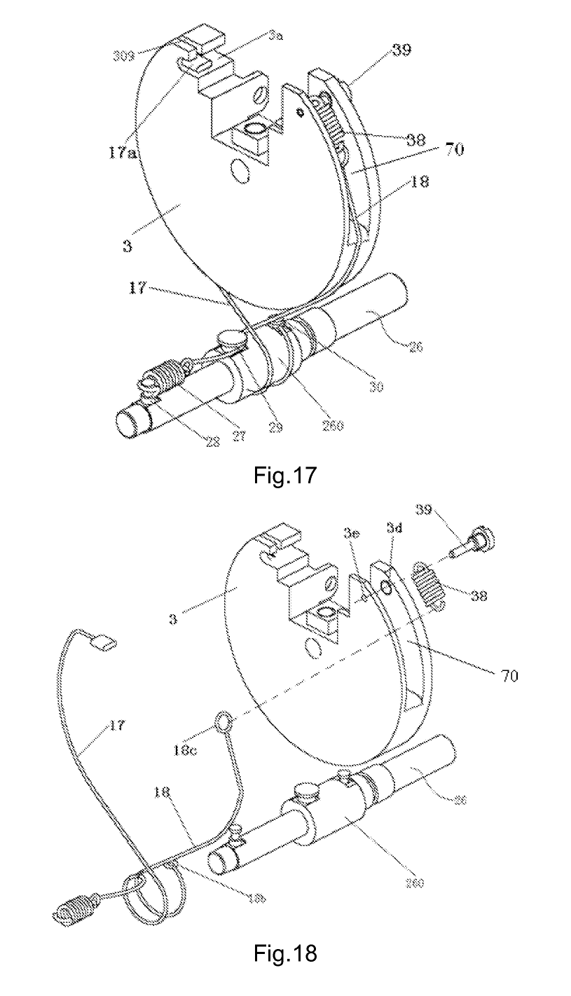

As shown in FIG. 17 and FIG. 18, an accommodating groove 70 is formed at the upper side of the driven wheel along the rim; a spring 38 is arranged in the accommodating groove 70; and a joint structure is arranged at the upper side of the accommodating groove 70. In the depicted embodiment, the joint structure comprises a pin 39, which is fixedly connected to the driven wheel 3 through the hole 3e and 3d on the sidewall of the accommodating groove 70. The hook ring on one end of the spring 38 hooks the pin 39, and another hook ring on another end hooks the connection end 18c of the rope 18 (which may be a ring). The rope 18 winds the driven wheel 3 for a certain distance, and winds the winding area 260 of the revolving shaft 26 for a certain distance. Another connection end 18b (which may be a ring) is clamped to the king pin 30 which is fixedly connected with the revolving shaft 26. The other parts may be identical or similar to corresponding parts of the aforesaid embodiments and shall not be repeated here.

In the depicted embodiment, two springs are used to tighten the ropes, which makes the force provided by each spring may be small. Thus the deformation of the springs may be reduced, which makes assembly easy. Additionally, the springs may be housed in the accommodating groove of the driven wheel and no extra space is occupied.

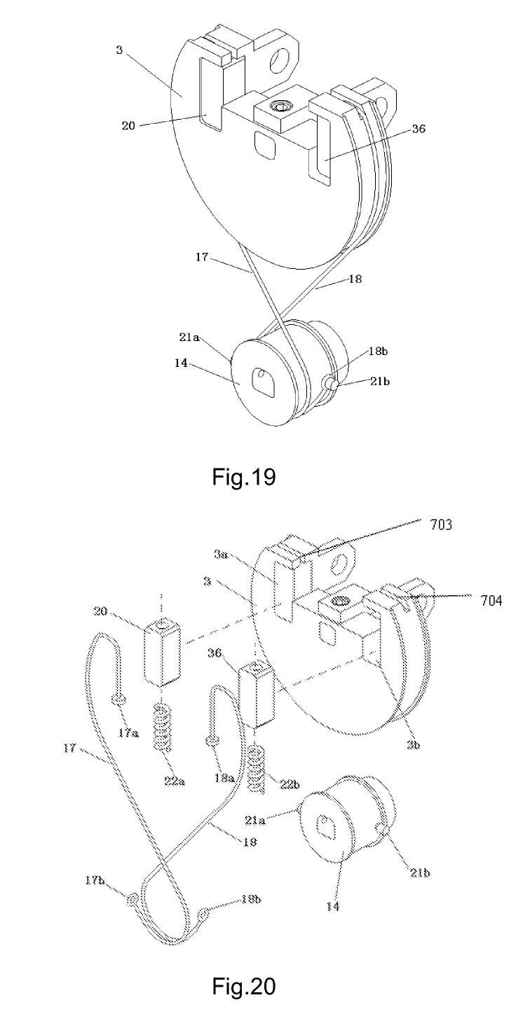

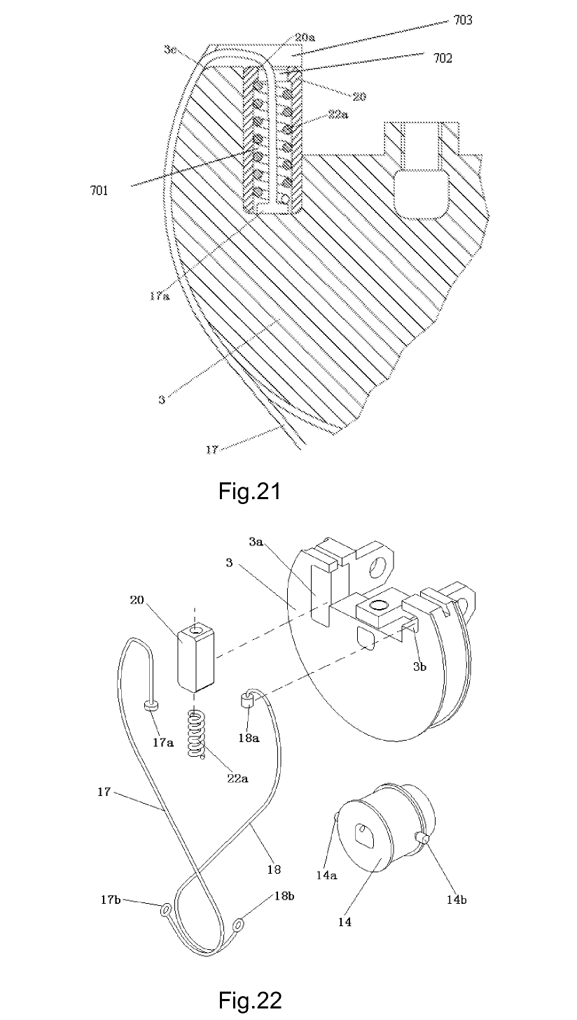

As shown in FIG. 19 to FIG. 21, the joint structure 3a and 3b of the driven wheel 3 may be accommodating grooves. A connection block 20 is housed in and matched with the joint structure 3a, and another connection block 36 is housed in and matched with the joint structure 3b. The connection block 20 may be cylindrical, cuboid or other shapes. As shown in FIG. 21, the connection block 20 comprises a hollow interior as a housing cavity 701. An opening is formed on one side of the connection block 20. A spring 22a may be housed in the housing cavity 701 through the opening. Take the side having the opening as the top side of the connection block 20 (that is, the housing cavity 701 is formed concavely at the tope side) and the side opposite to the top side as the bottom side. A hole 702, which is formed at the bottom side of the connection block 20, is connected with the housing cavity 701. Further, the diameter of the hole 702 is smaller than that of the spring 22a; thus, one end of the spring 22a may be propped against the bottom side of the connection block 20.

As shown in FIG. 21, the spring 22a is housed in the housing cavity 701 through the opening of the connection block 20. An end of the rope 17 passes through the hole 702 at the bottom side of the connection block into the housing cavity 701, and passes the spring 22a in the housing cavity. The connection end 17a (which may be a knot) at the end of the rope 17 is pressed at the end of the spring 22a, which is far away from the bottom side of the connection block 20.

As shown in FIG. 20, two pins 21a and 21b are formed on the driving wheel 14. Another end of the rope 17 passes through the groove 703 formed on the upper side of the joint structure 3a of the driven wheel 3 to the wheel surface of the driven wheel. The rope 17 winds the driven wheel for a certain distance and winds the driving wheel 14 for a certain distance. The connection end 17b is clamped to the pin 21a, which is fixedly connected to the driving wheel 14. In addition, as shown in FIG. 21, to reduce the damage to the rope, the corner 3c of the driven wheel 3 and the corner 20a of the connection block 20 may be rounded off.

The structure of the connection block 36 may be identical to that of the connection block 20 and the connection of the rope 18 may be identical to that of the rope 17. The connection block 36 may be housed in and matched with the joint structure 3b, and the spring 22b is housed in the housing cavity of the connection block 36. The rope, whose connection end 18a is pressed to an end of the spring 22b, passes through the spring 22b and the hole on the bottom side of the connection block 36, and winds the driven wheel 3 and the driving wheel 14 for a certain distance respectively. The connection end 18b on another end of the rope is clamped to the pin 21b which is fixedly connected to the driving wheel 14.

The springs 22a and 22b are compression springs in the depicted embodiment. One end of a compression spring is against to the bottom side of the connection block and another end is pressed by the connection end of a rope. Thus the force to tighten the rope may be offered by the elastic force of the compressed spring. Furthermore, in the illustrated embodiment, the connection block, in which the spring is housed, is housed in the driving wheel, thus the space occupied by the spring and the connection block is contained within the space occupied by the driven wheel. No extra space is needed. In this way the space occupied by the probe may be reduced.

In the depicted embodiment, only one spring may be used. As shown in FIG. 21, a similar structure may be used except that the connection block 36 and spring 22b are not used herein and the connection end 18a of the rope 18 is directly connected to the joint structure 3b of the driven wheel 3. In this embodiment, the rope may be tightened continuously by adopting only one spring.

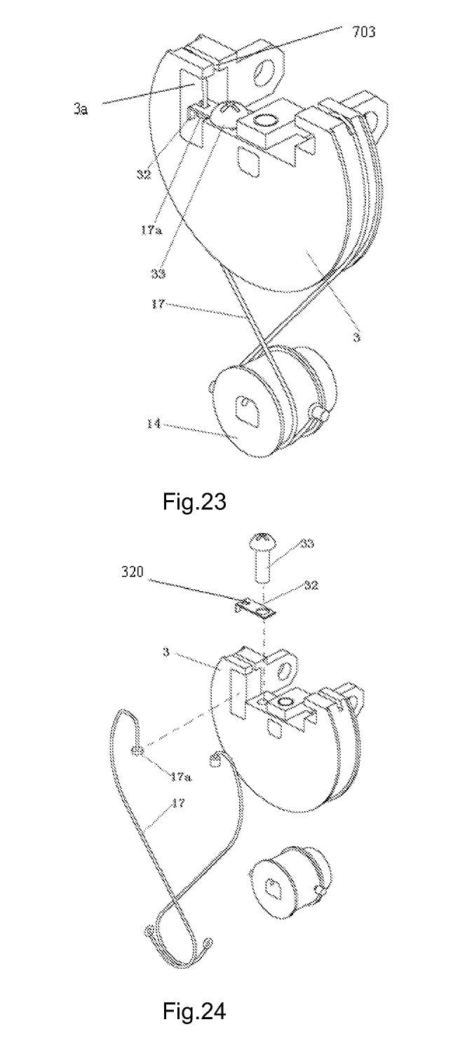

In other embodiments, the springs may be replaced with other elastic parts, such as elastic slices, as shown in FIG. 23 to FIG. 25. Referring to FIG. 23 and FIG. 24, the joint structure 3a has a spring piece 32 instead of link block and spring. Instead of connection blocks and springs, an elastic slice 32 is arranged on the joint structure 3a. A connection end 320 is arranged on one end of the elastic slice 32. The connection end 320 extends into the groove of the joint structure 3a, while another end of the elastic slice 32 is fixedly connected to the driven wheel 3 through the screw 33. The connection end 17a of the rope 17 passes through the groove 703 at the upper side of the joint structure 3a of the driven wheel 3, enters the groove of the joint structure 3a, and is connected to the connection end 320 of the elastic slice 32. The other parts and the winding of the rope in the embodiment are identical to those in the last embodiment.

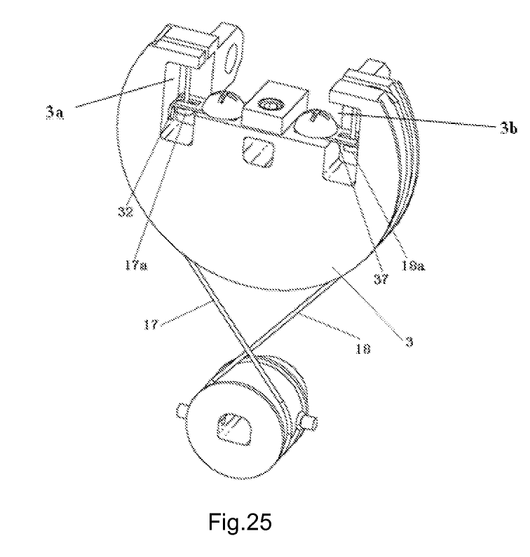

As shown in FIG. 25, the connection blocks and springs at the joint structures 3a and 3b are replaced with the elastic slices. The other parts in the embodiment are identical to those described above and shall not be repeated here.

Though the above embodiments describe the invention in detail, the invention is not limited by these specific embodiments. It will be understood by those having skill in the art that many changes may be made to the details of the aforesaid embodiments without departing from the underlying principles of the invention.

What is claimed is:

1. An ultrasonic probe, comprising:
a base;
a motor fixedly connected to the base;
a driving wheel connected to an output of the motor and driven by the motor;
a driven wheel rotationally connected to the base with a shaft;
a transducer fixedly connected to the driven wheel for emitting and receiving ultrasonic echoes;
two ropes, both ends of each rope having a connection end respectively; one connection end on one end of each rope being respectively connected to the driving wheel and another connection end on another end of each rope being respectively connected to the driven wheel; and
at least one elastic part through which the connection end of at least one end of at least one rope is connected to the driving wheel or the driven wheel in a buffering manner, wherein one end of the elastic part is connected to the connection end of the rope, and another end of the elastic part is connected to the driving wheel or the driven wheel.

2. The ultrasonic probe of claim 1, wherein the driving wheel is provided with at least one joint structure, and the end of the elastic part is connected to the joint structure.

3. The ultrasonic probe of claim 1, wherein at least one boss is formed on the driving wheel, one end of the elastic part being fixedly connected to the boss with a screw and another end of the elastic part being provided with a joint structure which is connected with the connection end of the rope.

4. The ultrasonic probe of claim 1, wherein at least one gap is formed on the rim of the driving wheel through which the rope is connected to the end of the elastic part.

5. The ultrasonic probe of claim 1, wherein the driven wheel is provided with at least one joint structure, and the connection end of the rope which is connected with the driven wheel is connected to the joint structure.

6. The ultrasonic probe of claim 1, wherein the driving wheel comprises a shaft which is rotationally connected to the base, and wherein the connection end of the rope which is connected to the driving wheel is connected to the shaft.

7. The ultrasonic probe of claim 6, wherein a link pin is arranged near one end of the shaft, and wherein the end of the elastic part is connected to the link pin.

8. The ultrasonic probe of claim 6, wherein one end of the elastic part is fixedly connected to a place near one end of the shaft with a screw, and another end of the elastic part is provided with a joint structure which is connected with the connection end of the rope.

9. The ultrasonic probe of claim 6, wherein the probe comprises at least two elastic parts, wherein one end of one the elastic part is connected to the shaft, and another end of the elastic part is connected to the connection end of an end of one rope, while one end of another elastic part is connected to the driven wheel, and another end of the another the elastic part is connected to the connection end of an end of another rope.

10. The ultrasonic probe of claim 9, wherein a housing groove which is provided with a joint structure is formed on the driven wheel, and wherein the elastic part which is connected to the driven wheel is housed in the housing groove and connected to the joint structure.

11. The ultrasonic probe of claim 7, wherein the shaft is provided with a king pin whose location at the shaft is farther away from end of the shaft than the link pin at the shaft; and the king pin is bypassed by at least one the rope.

12. The ultrasonic probe of claim 1, wherein at least one housing groove is formed on the driven wheel, a connection block being respectively housed in each housing groove; wherein the connection block comprises a top side which concavely forms a housing cavity and a bottom side which is provided with a hole; wherein the elastic part is a spring housed in the housing cavity; wherein one connection end of at least one the rope is clamped to one end of the spring and the rope extends to the housing groove through the spring and the hole at the bottom side of the connection block; and wherein another end of the spring is propped against the bottom side of the connection block.

13. The ultrasonic probe of claim 1, wherein at least one housing groove is concavely formed on the driven wheel; wherein at least one end of the elastic part is fixedly connected to the driven wheel with a screw, another end of the elastic part which is provided with a connection end being extended into the housing groove; and wherein the connection end of one end of at least one rope is connected with the connection end of the elastic part.

14. The ultrasonic probe of claim 12, wherein the driving wheel is provided with a pin, the connection end of the rope which is connected to the driving wheel being connected with the pin.

15. The ultrasonic probe of claim 13, wherein the driving wheel is provided with a pin, the connection end of the rope which is connected to the driving wheel being connected with the pin.

16. The ultrasonic probe of claim 1, wherein the ropes are wound on the driven wheel and the driving wheel in opposite winding directions.
